(19) United States
(12) Patent Application Publication
(10) Pub. No.: US 2005/0287515 A1
(43) Pub. Date: Dec. 29, 2005

(54) Removal of bacterial DNA from therapeutic fluid formulations

(76) Inventors: Reinhold Deppisch, Hechingen (DE); Werner Beck, Rottenburg (DE); Ralf Schindler, Berlin (DE)

Correspondence Address: GREENLEE WINNER AND SULLIVAN P C, 4875 PEARL EAST CIRCLE, SUITE 200, BOULDER, CO, 80301, US

(21) Appl. No.: 11/100,941

(22) Filed: Apr. 7, 2005

Related U.S. Application Data
(60) Provisional application No. 60/583,226, filed on Jun. 24, 2004.

(51) Int. Cl. A01N 1/02

(52) U.S. Cl. 435/2; 530/412

(57) ABSTRACT

This invention discloses methods and devices useful for eliminating or reducing the adverse effects associated with the use of a variety of therapeutic fluids. It is proposed herein that the source of such side effects (e.g. fever and septic shock) is the oligonucleotides present in the fluid and that using the ultrafiltration and adsorption methods and devices disclosed in the invention one can minimize the risk of these adverse effects often associated with dialysis treatment.

### CROSS-REFERENCE TO RELATED APPLICATIONS

This application claims benefit of provisional application No. 60/583,226 filed Jun. 4, 2004, which is incorporated herein to the extent not inconsistent herewith.

### BACKGROUND OF THE INVENTION

The present invention is concerned with a fluid for medical use. More particularly, the present invention relates to methods and devices to remove oligonucleotides present in various therapeutic fluids.

In the human body, solutes transfer from one body fluid to another by diffusion processes which include dialysis, osmosis and ultrafiltration. Unwanted solutes, toxins and excess water are transferred from the blood stream by dialysis in the kidneys for excretion from the body. In the event of kidney malfunction, haemodialysis is often used for this function as well as kidney transplantation.

Haemodialysis is based on the principle of allowing blood to contact a semipermeable membrane, the other side of which is in contact with an isotonic dialysis solution. Toxins and other relatively small molecules, diffuse across the membrane until their concentration equilibrates in both the dialysis solution and the blood. The isotonic dialysis solution is then changed to a fresh solution to permit continued purification of the blood. Solution replacement may be continuous or discontinuous. The efficiency of dialysis is directly related to many factors including, volume of dialysis fluid, number of changes of dialysis solution (or flow rate in a continuous system), length of time between changes, surface area of membrane, pore size of the membrane, rates of diffusion of the toxins, etc.

Peritoneal dialysis is carried out by substituting the artificially provided semipermeable membranes of the haemodialysis machine with natural semipermeable capillary bed membranes that are abundant within the peritoneal cavity. By continuously flooding the interperitoneal space with isotonic dialysis solution, exchange of toxins from the blood occurs and dialysis is accomplished.

There have been steady improvements in the field of dialysis with respect to the apparatus as well as the compositions of the fluid. However, there are still many problems associated with the procedure. Of those, a major concern is various adverse effects associated with microbial contamination of the dialysis fluid [Yamagami et al., (1990) Int J Artif Organs 13(4):205-210]. Endotoxins, particularly those of bacterial origin, are known to stimulate production of various cytokines which can cause fever and septic shock. Previous studies have linked these problems with lipopolysaccharides and muramyldipeptides. However, removal of such compositions does not entirely eliminate these adverse side effects.

Therefore there is an urgent and continuing need to improve the safety of therapeutic fluids including dialysis fluids in order to reduce the incidence of adverse side effects associated with their use. Towards this goal, the inventors herein provide methods and devices for purifying therapeutic fluids prior to use, which will minimize such adverse side effects. This invention is based on the finding that the therapeutic fluids currently in use are contaminated with oligonucleotides of microbial origin, even after the application of currently approved purification methods. It is proposed herein that the oligonucleotides present in the fluids are the cause for the observed adverse effects.

The importance of oligonucleotides as a source of chronic and acute inflammatory stimulation is underscored by the fact that lipopolysaccharides and bacterial DNA can act synergistically to stimulate the release of pro-inflammatory cytokines, such as TNF alpha, by macrophages [Gao et al. (2001) J Immunol; 166: 6855-6860]. The results shown in FIG. 14 further confirm these studies.

Another problem associated with the bacterial contaminants is sub-acute chronic inflammation. Cardiovascular complications are the major cause of mortality in hemodialysis patients and peritoneal dialysis patients. More than 50% of these patients die from cardiovascular causes [United States Renal Data System, Annual Report (2003), Am J Kidney Dis. (2003) December;42(6 Suppl. 5)] and atherosclerosis is one of the major underlying conditions leading to these complications. The development of atherosclerosis is strongly related to inflammatory processes taking place at the surface of blood vessels [Ross New Engl J Med (1999); 340: 115-126]. Cellular as well as plasmatic activation mechanisms are involved in these inflammatory processes. The process is known to start when bacteria or bacterial products enter the blood stream, by which white blood cells become activated. These activated cells start to form adhesion molecules on their surface, which make these cells adhesive or “sticky”. They can now interact with the cells at the surface of the blood vessels, the endothelial cells, where they can cause tissue damage (Springer Nature. 1990; 346: 425-434; Ley Cardiovasc Res. 1996; 32: 733-742). At the same time the activated white blood cells start to release cytokines, for example, interleukin 6, into the blood stream, which are then transported with the blood to other perfused tissues, for example, the liver. In the liver the so-called acute phase reaction is triggered by these cytokines. This acute phase reaction leads to profound changes in the protein synthesis of the liver [Gabay C et al. (1999) N Engl J Med; 340: 448-454]. Some proteins are synthesized more (positive acute phase proteins, such as C-reactive protein or fibrinogen), some proteins are synthesized less (negative acute phase proteins, such as serum albumin and transferrin). The level of C-reactive protein (CRP) rises very fast after an acute inflammatory insult to very high levels (more than 1000 fold above normal), but usually goes down within a few days when the cause of the inflammation has been resolved. Micro-inflammation is characterized as chronic inflammation on a sub-clinical level. This can be described, for example, by the CRP-level. CRP levels of healthy people are normally below 5 mg/l, often below 1 mg/l. During an acute inflammatory period, for instance, during a bacterial infection, when clinical signs of inflammation are visible, CRP is usually far above 50 mg/l. It is known from large studies in the general population (Ridker et al. N Engl J Med 1997; 336: 973-979) that chronic inflammation as indicated by elevated CRP (above 1 mg/l) is associated with an increased risk of cardiovascular disease and an increased risk of developing diabetes. In chronic renal failure patients such elevated CRP is strongly linked to malnutrition, atherosclerosis and mortality (Stenvinkel et al. Kidney Int 1999; 55: 1899-1911) and in dialysis patients elevated CRP correlates with mortality (Zimmermann et al. Kidney Int 1999; 55:648-658). These studies emphasize the importance of removing any contaminating oligonucleotides of microbial origin from the therapeutic fluids before administering to patients. The advantages of the present invention will become apparent from the following description.

### SUMMARY OF THE INVENTION

The present invention provides methods and devices useful for eliminating or reducing the adverse effects associated with introducing various forms of therapeutic fluids into animals, particularly humans. This invention is based on the finding that the therapeutic fluids currently in use contain various amounts of oligonucleotides of microbial origin and that such oligonucleotides can cause adverse effects such as fever and septic shock by stimulating production of various cytokines when administered in vivo. Accordingly, these findings led the inventors to develop methods and devices for removing the oligonucleotides present in the therapeutic fluid. The therapeutic fluid as used herein includes dialysis fluid, infusion fluid, any other forms of fluid which are intended to be introduced into animals, preferably humans, for medical use. Thus, it includes any body fluid, but is not limited to blood and cerebrospinal fluid, water and any fluid prepared from cell culture which eventually becomes part of therapeutic fluid.

The contaminant oligonucleotides are generally double stranded deoxynucleotides, which originate largely from bacteria and other microorganisms. These oligonucleotides can range in size from as little as about 5 nucleotides and larger (up to about 500 nucleotides). The contaminant oligonucleotides can also be single stranded deoxynucleic acids or ribonucleic acids of similar size. The contaminant oligonucleotides may further be complexed to other compounds such as proteins, peptides, metal ions, fatty acids, amino acids, and phospholipids.

The contaminating oligonucleotides can be removed by a variety of means, preferably by ultrafiltration and/or adsorption. Preferably, these means carry out a selective removal of oligonucleotides while retaining other necessary compositions in the fluid.

The ultrafiltration is carried out by passing the fluid through a membrane which can separate a low molecular weight DNA (e.g. oligonucleotides as small as 5-10 bp). Further, the oligonucleotides can also be removed with an ultrafiltration membrane comprising cationic charged materials, such as PAES and PEI having cationic charges (blend of polyarylethersulfone and polyethyleneimine and/or modified polyethyleneimine), or PAES and PPO having cationic charges (blend of polyarylethersulfone and polyphenyleneoxide and/or modified polyphenyleneoxide). With these types of membranes you have the possibility to combine size exclusion as well as absorption of the oligonucleotides.

The oligonucleotides can also be removed by adsorbing onto beads comprising polystyrene grafted with polyethyleneglycol containing polyarginine or ARG-8 like the ones disclosed in WO 01/23413 or WO 2004/004707, which hereby are incorporated by reference. The glass composite particles, collagen coated matrices, nets and meshes coated with collagen can also be used as adsorbing means.

The invention further provides devices comprising the ultrafiltration and/or adsorption means described above singly or in combination (i.e., multiple untrafiltration and/or multiple adsoption means) for carrying out a selective removal of the contaminant oligonucleotides from the therapeutic fluids.

The processes and devises of the invention are intended to remove at least about 50% of the contaminating oligonucleotide present in a given therapeutic fluid, preferably about 70%, most preferably about 90%. The percentage is a fraction of the oligonucleotide removed by the processes or devices of the invention compared to the amount of the oligonucleotide present in a given therapeutic fluid before the step of removing.

### BRIEF DESCRIPTION OF THE DRAWINGS

FIG. 1 is a photo of agarose gel electrophoresis of DNA present in the supernatants of three different Pseudomonas culture (Ps 1-3). The gel was stained with ethidium bromide for visualization. The arrow indicates the short DNA fragments and the band shown on top is high molecular weight DNA.

FIG. 2 shows that the supernatants of three different bacterial culture (E. coli, Pseudomonas Malto and Enterok. faec.) contain small DNA fragments smaller than 20 bp in size.

FIG. 3 shows that the dialysates tested contain short DNA fragments. The dialysates labeled 1-6 were obtained from six different dialysis machines routinely used in the art. DNA was extracted with C18 column and labeled using digoxygenin.

FIG. 4 demonstrates that an ultrafiltration step using polysulfone or polyamide hollow fiber membranes can reduce the amount of short DNA fragments present in the supernatant of Pseudomonas culture, standard dialysate fluids 1-4, and purified water. The lanes labeled as “pre” and “post” indicate the amount of DNA present before and after the step of ultafiltration.

FIG. 5 is a scheme showing the application of the invention, i.e., a column comprising the beads for adsorption can be placed such that a given therapeutic fluid can be filtered to remove contaminant oligonucleotides before it is administered into a subject.

FIG. 6 is a scheme showing the cross-sectional view of a hollow membrane which is selectively treated outside for removing oligonucleotides from the therapeutic fluid.

FIG. 7 further shows that dialysate samples, RO-water-samples (i.e. the water distributed by a pipe system in a dialysis clinic just before it enters the dialysis machine), saline for intravenous infusion, and various bacterial cultures contain different amounts of small DNA fragments. ODN were detected in 18 of 20 investigated dialysate samples (two representative samples are shown), in 8 of 10 reverse-osmosis water samples (two representative samples are shown) and in all cultures from various bacterial strains. The presence of bacterial DNA in dialysate was confirmed by PCR specific for bacterial tRNA gene sequences.

FIGS. 8A-8C illustrate that the use of modified inorganic particles (e.g., beads) to remove oligonucleotides present in the pseudomonas culture supernatant reduced the induced levels of IL-1β and IL-6 by the culture supernatant.

FIG. 9 is a scheme showing the principle of the hemodialysis process, i.e. a fluid circuit contained in a dialysis machine and the blood circuit with a dialyzer connected to a patient. RO water coming from a distribution pipe enters the dialysis machine, where electrolytes are admixed (either from liquid concentrates or solid powders) to prepare the dialysate. Dialysate is then pumped through the dialysate compartment of the dialyzer. On the blood circuit side patient's blood is pumped through the blood compartment of the dialyzer and returned to the patient. The circles indicate the possible locations of means of the invention to reduce the DNA content of water or dialysate

FIG. 10 shows how an additional means of purification according to the invention for removing the contaminating ODNs from the fluid can be added to a manual PD-system. X indicates preferred location and Y indicates an alternative location for such means (e.g. ultrafiltration and/or adsoprtion).

FIG. 11 is an example of how an additional means according to the invention of purification for removing the contaminating ODNs from the fluid can be added to an automated PD-system. X indicates preferred location and Y indicates an alternative location.

FIG. 12 is another example of the use of the purification means based on the invention. X indicates the location of the means. The types of infusion fluid that can be provided in the bag include, but are not limited to saline, buffer solutions, cell culture media, recombinant protein solutions, substitution fluids for hemofiltration or hemodiafiltration, serum albumin, coagulation factors, immunoglobulins, parenteral or intravenous nutrition fluids, fresh frozen plasma, blood substitutes (e.g. modified hemoglobin), plasma expanders, and cell concentrates (e.g. erythrocytes, platelets, therapeutic cell products).

FIGS. 13A and 13B are examples of two water preparation systems for which the devices of the invention can be applied to remove contaminating ODNs from the water. FIG. 13A is an example of the water preparation system for homes and hospitals and FIG. 13B exemplifies a water preparation system for dialysis clinics with the added means for removing ODNs according to the invention.

FIG. 14 shows that synthetic bacterial DNA (CpG) and LPS stimulated TNF alpha release when incubated with isolated whole blood in contrast to non-CpG DNA (non-bacterial). When both of these agents (CpG+LPS) were applied together, the stimulation of TNF alpha release was significantly enhanced.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

The term “therapeutic fluid” as used in the invention refers to any fluid which is intended to be administered into human patients or animals for a therapeutic purpose. “Therapeutic fluid” may include a fluid for dialysis, hemofiltration, hemodiafiltration, infusion therapy, lavage procedure etc. The most common type of therapeutic fluid is a fluid for dialysis. “Dialysis” is a form of transport of molecules between two compartments of separate compositions using a barrier such as a membrane. Haemodialysis and peritoneal dialysis are examples of the type of dialysis and they can be continuous or noncontinuous.

The term “oligonucleotide or ODN” as used herein generally refers to a double stranded deoxynucleic acid molecules of at least about 5 bp up to about 500 bp, preferably in the range of about 5-200 bp, and used in some cases synonymously with “small DNA fragment”. The contaminating oligonucleotides can also be single stranded deoxynucleic acid molecules and in some instances ribonucleic acid molecules of similar size. Typically, the contaminating ODNs are of microbial origin. The term, “microbial origin” is used herein to indicate that the source of the ODNs present in a given therapeutic fluid is generally a bacterium which includes, but is not limited to, the following species (sp): Escherichia coli, Pseudomonas sp, Enterococi, Moraxella sp, Alcaligenes sp, Flavobacteria sp, Acinetobacter sp, Flavobacteria sp, Serratia sp, Klebsiella sp, Enterobacter sp, Bacillus sp, Mycobacteria sp, Corynebacterium sp, Micrococcus sp, Staphylococcus sp, Streptococcus sp, Achromobacter sp, Aerobacter sp, Erwinia sp, Aeromonas sp, and Xanthomonas sp, However, the oligonucleotides present in a therapeutic fluid can also be derived from any single celled organism such as viruses, fungi, yeasts, molds, algae, and amoebae.

The term “ultrafiltration” as used herein refers to a process of selectively removing or preventing any undesired substance from moving from one side of the device (e.g. a membrane) to the other side. The ultrafiltration process is controlled according to the parameters determined by the type of the membranes used and the pore size thereof. An ultrafiltration membrane normally has a pore size within the range of 0.001 to 0.01 μm. For example, the membranes are comprised in such a way that the substance is moved across the membrane according to the size. The membranes useful for ultrafiltration in the present invention can be modified and comprised of in such a way that they serve a selective filtering function, i.e., selective removal of the oligonucleotides. In one embodiment the oligonucleotides are removed with an ultrafiltration membrane comprising cationic charged materials, such as polyarylethersulphone (PAES) and polyethyleneimide (PEI) having cationic charges (blend of PAES and PEI and/or modified PEI), or PAES and polyphenyleneoxide (PPO) having cationic charges (blend of PAES and PPO and/or modified PPO).

The term “adsorption” as used herein refers to a process of removing any substance from a fluid by mixing the fluid with an adsorbing material (i.e. beads or particles such as silica particles). The beads or particles are comprised of a specific material, e.g. as described in WO0123413 and WO2004-004707 with a size of 100 nm to 500 μm in particle diameter so that they can bind to the oligonucleotides and other impurities of similar size

The present invention relates to a major problem often encountered in the field. The patients who undergo dialysis treatment often have serious side effects such as septic shock. Various attempts to identify the cause and preventive measures for this have not been entirely successful. It is proposed herein that the oligonucleotides present in the currently available therapeutic fluids are the cause for the adverse effects seen in these patients. This is consistent with the data disclosed herein that the ultrafiltration membranes currently used for dialysate preparation and water purification are not adequate for removing contaminant oligonucleotides and that the oligonucleotides of microbial origin can induce cytokine production. Therefore, the present invention provides methods and devises to remove the oligonucleotides present in a variety of therapeutic fluids to eliminate or minimize the adverse effects observed in patients receiving such fluids.

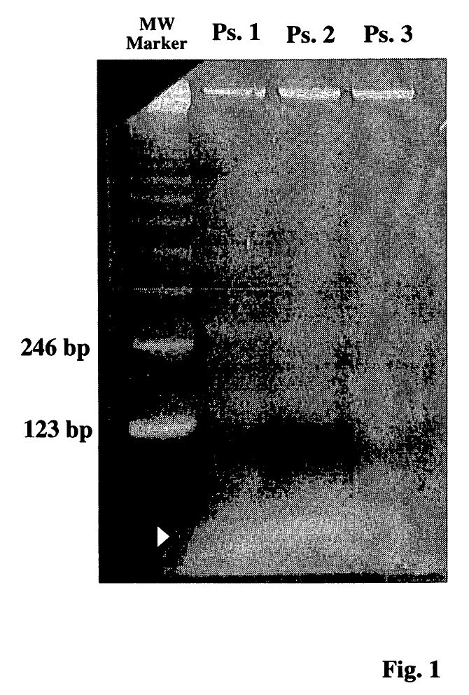

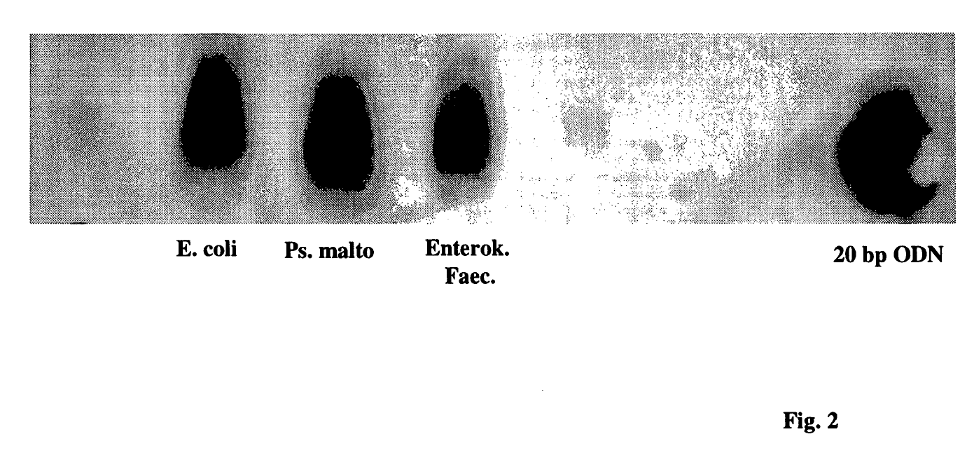

To demonstrate the presence of short DNA fragments (i.e. oligonucleotides) in various culture supernatants, DNA was extracted from Pseudomonas culture supernatants using a reverse phase column (e.g. Sepac C18). For each experiment, 15 to 500 ml bacterial supernatant was applied to the column and the column was then rinsed sequentially with acetonitrile, distilled water, and ammonium acetate. DNA bound in the column was eluted with 2 ml of 60% methanol, precipitated with ice cold ethanol, and dried under vacuum to concentrate. DNA was quantitated by measuring adsorption at 260 and 280 nm, respectively. As shown in FIG. 1, all three supernatants from Pseudomonas culture contain a significant amount of DNA below the size of 100 bp. The results shown in FIG. 2 further demonstrate that the culture supernatants of Pseudomonas malto, E. coli, and E. faecalis contain significant amounts of oligonucleotides.

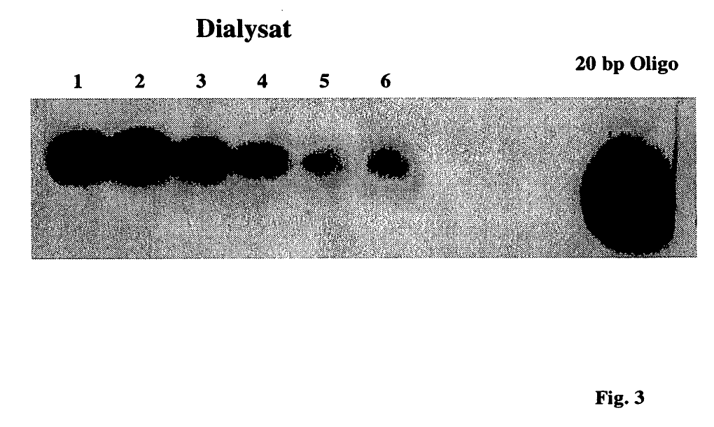

Oligonucleotides can also be detected in various dialysates that are currently in use. DNA was extracted from six dialysates obtained from six independent dialysis machines which are used routinely in the art. DNA was prepared as described above and labeled with digoxigenin (DIG) using the DIG-oligonucleotide labeling kit according to the manufacturer's instructions (Boeringer Mannheim, Germany). The enzyme, terminal transferase in the kit, can label DNA fragments of various sizes (10 to 100 bp) with high specificity. The labeled products were separated on a 2% agarose gel, which was then transferred to nylon membranes by capillary blotting. The nylon membranes were processed using the DIG-oxygenin detection kit (Boeringer Mannheim, Germany) and exposed to Biomax X-ray films (Kodak, Rochester, N.Y.). The results are shown in FIG. 3. It is clear that all six dialysates contain different but significant amounts of oligonucleotides.

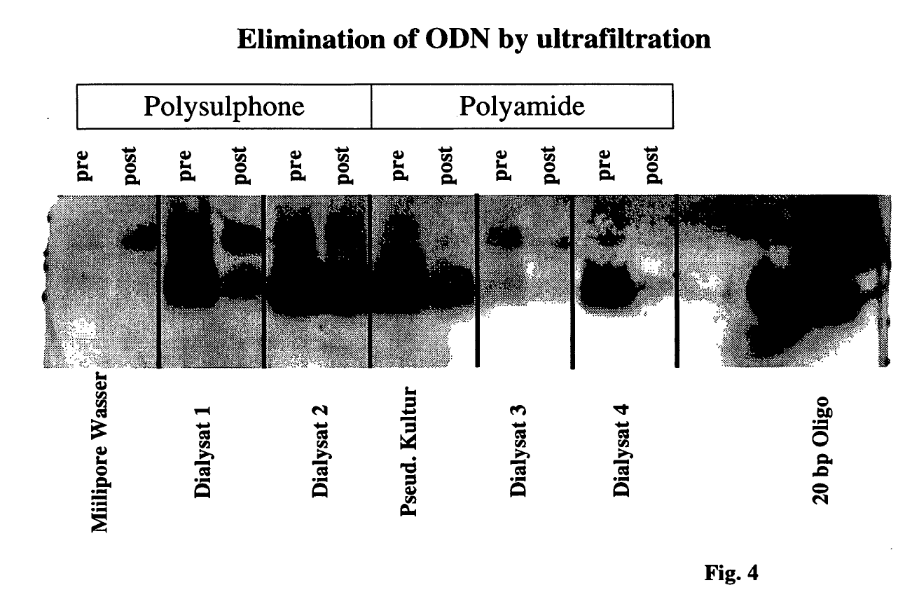

In order to test whether the oligonucleotides detected in the culture supernatants and dialysates can be removed by the dialysis membranes currently in use, the following experiments were carried out. The supernatants of Pseudomonas culture, standard dialysis fluids and the water purified using Millipore water purification system were filtered through hollow fiber membranes of either polysulfone or polyamide. The samples were taken before and after the filtration step to determine the level of the oligonucleotides. As shown in FIG. 4, the oligonucleotides are still present at significant levels after the ultrafiltration step in all the samples analyzed

The experiments described above clearly demonstrate that the filtration methods used currently to prepare water and dialysis fluids are not sufficient to remove small DNA fragments present as contaminants.

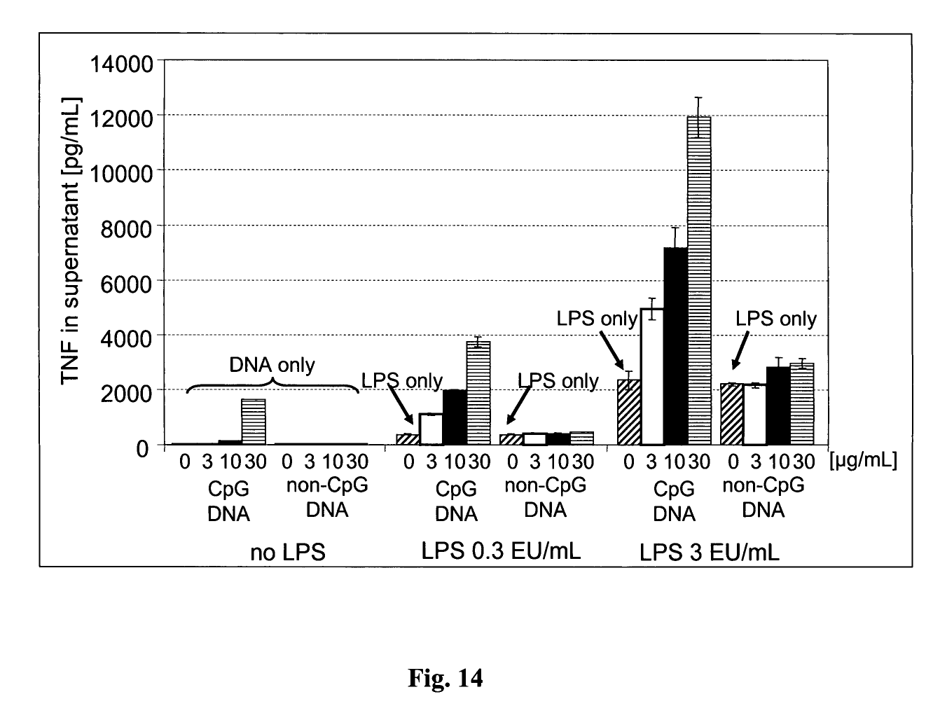

To further establish that the contaminant oliogonucleotides of bacterial origin are indeed able to induce cytokine release, heparinized human whole blood (800 μl) was incubated with LPS solutions (100 μl, containing LPS concentrations 0, 0.3 or 3 EU/ml) at 37° C. After 2 h, 100 μl of synthetic bacterial DNA (CpG) or non-bacterial (non-CpG) DNA was added at concentrations of 3 to 30 μg/ml. 1 ml 0.1 M EDTA was added after 6 h incubation at 37° C., the cells were centrifuged at 2000 g at 4° C. for 20 min and the supernatant was taken for analysis of TNF alpha by ELISA. The results shown in FIG. 14 clearly establish that the oligonucleotides as well as LPS stimulate TNF alpha release and that the combination of the two has enhanced effects on TNF alpha release. These results indicate that the presence of the oligonucleotides in the therapeutic fluids is likely the causative agent for the adverse effects seen in dialysis patients.

The contaminant oligonucleotides can be removed by an improved ultrafiltration or adsorption step. This improved step comprises a means (e.g., hollow fiber membrane) specifically designed to remove oligonucleotides of approximately 5-10 bp. Because of the small size of the oligonucleotides, the pore size either in an ultrafiltration or adsorption means should be as small as approximately 10 nm or smaller. The ultrafiltration membranes can also be comprised of nano-particles based on glass composites and include any membranes with outside plasma modification. Any meshes or nets which have been treated with collagen for the purpose of filtration are also within the scope of the invention. The contaminant oligonucleotides can also be removed by a variety of adsorption means that are known in the art. These include, but are not limited to, polystyrene beads grafted with polyethyleneglycol, polyarg (polyarginine, linear and branched oligo-arginine) or ARG-8 or glass composite particles or matrices coated with collagen.

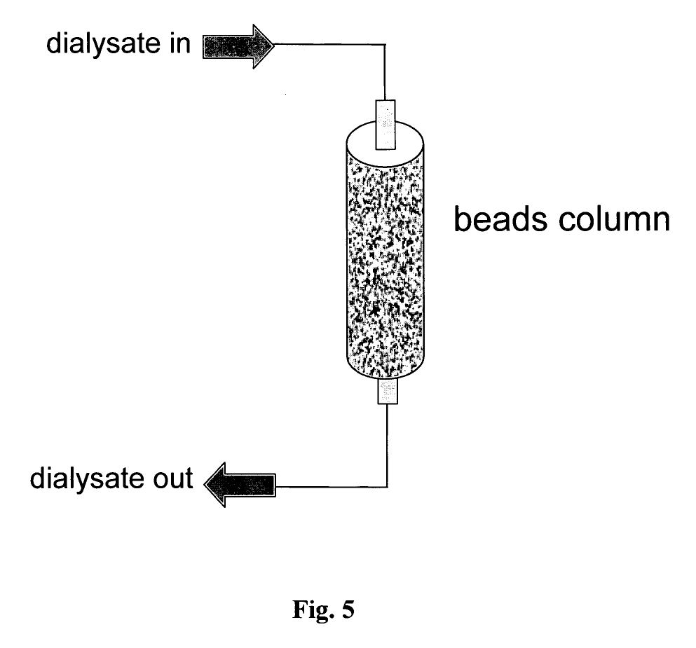

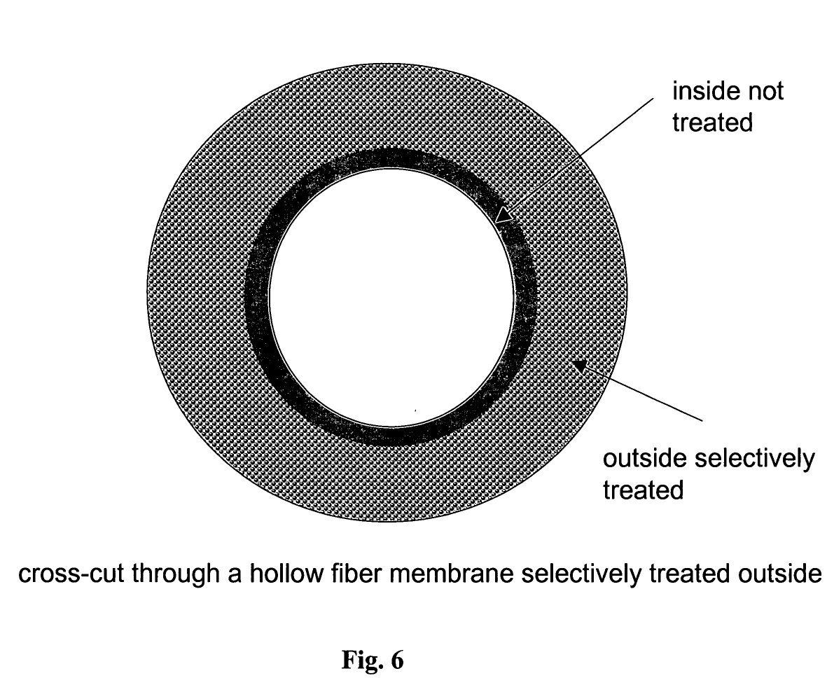

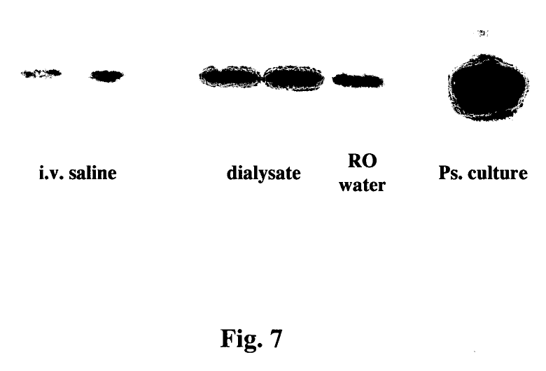

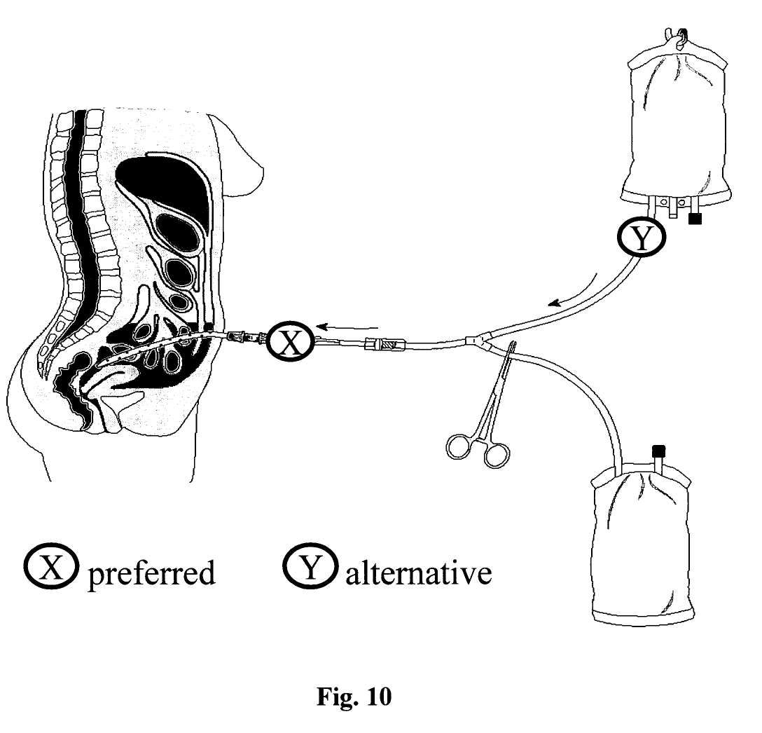

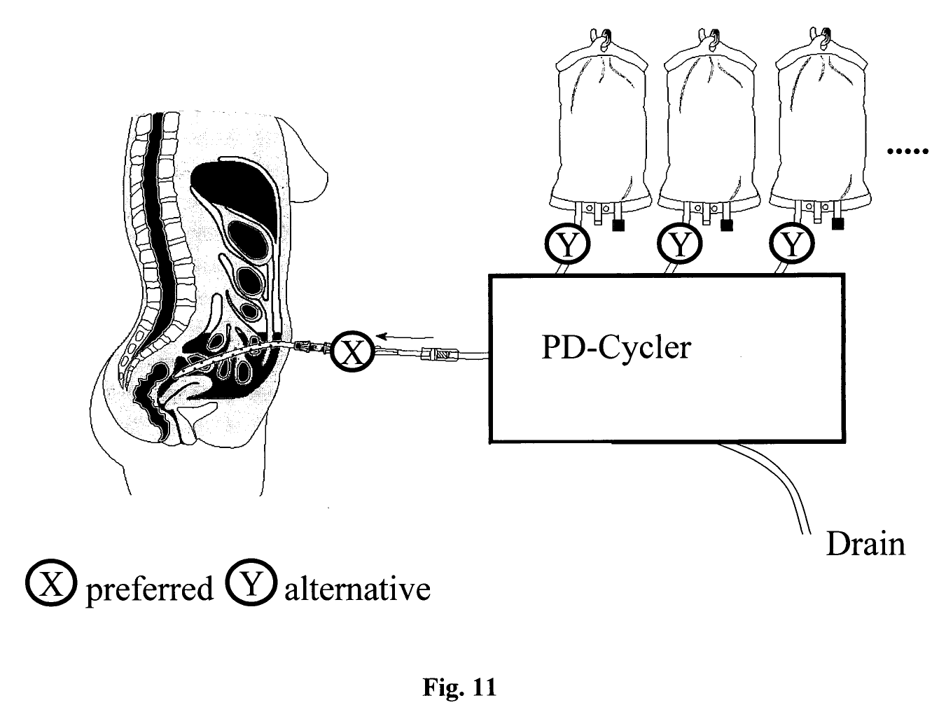

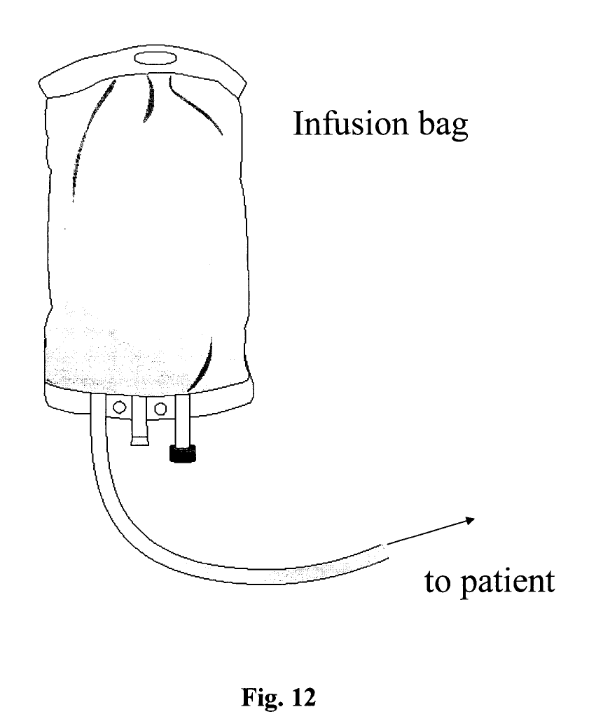

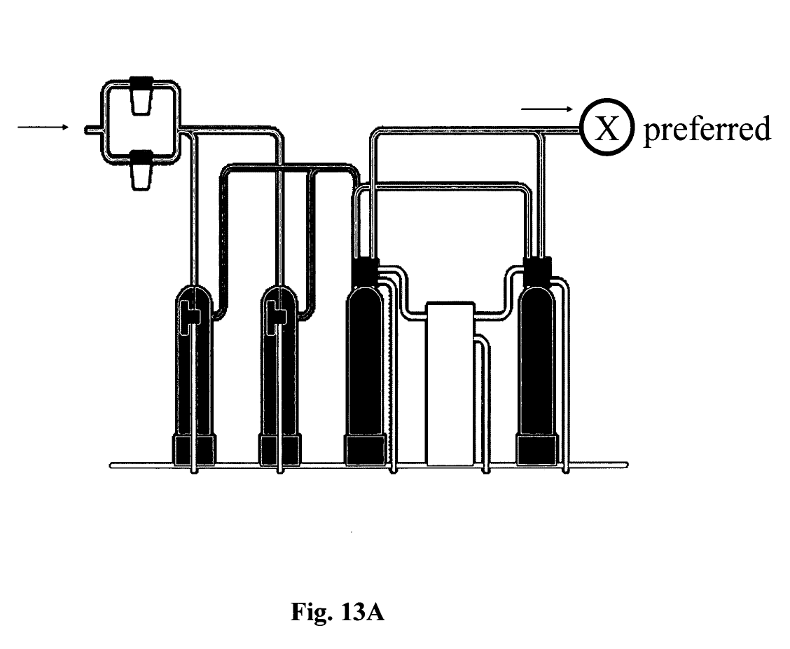

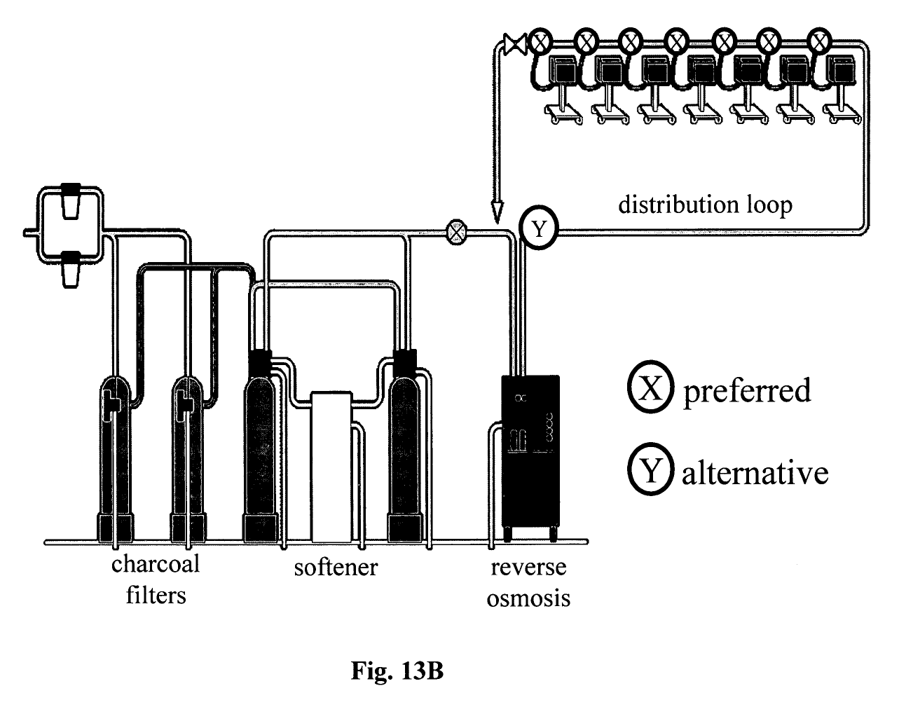

The ultrafiltration and adsorption means disclosed herein can be incorporated into a device (s) that can be used to remove the oligonucleotides in the therapeutic fluids prior to administering to the patients. FIGS. 5 and 6 are schemes illustrating the concepts of the invention. Examples of the application of the device(s) of the invention are depicted in the drawings (FIGS. 10-13).

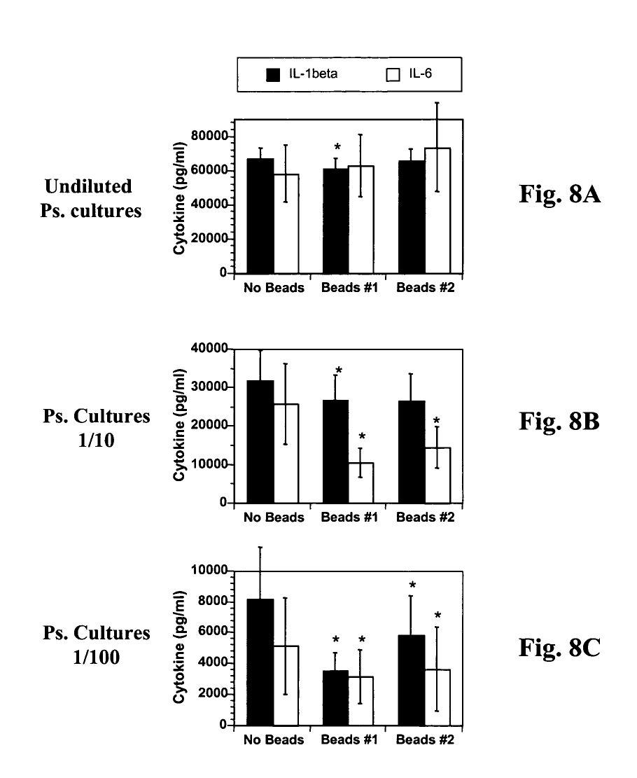

FIGS. 8A-8C illustrate that the supernatant of Pseudomonas culture induces cytokine release (IL-1β and IL-6) when undiluted regardless of the treatment with beads. However, when the supernatant is diluted ( 1/10 or 1/100), the levels of cytokine release are decreased after the treatment with the beads. Details of this experiment are provided below in the Examples Section. The beads used for these experiments are magnetic glass particles containing a magnetic core and an outer glass layer as described in U.S. Pat. No. 6,255,477. These beads are known to be useful for separating biological material such as nucleic acids. These results further support the invention, i.e., the removal of the contaminating oliogonucleotides in the therapeutic fluids can reduce or eliminate the adverse effects such as fever and septic shock associated with dialysis treatment.

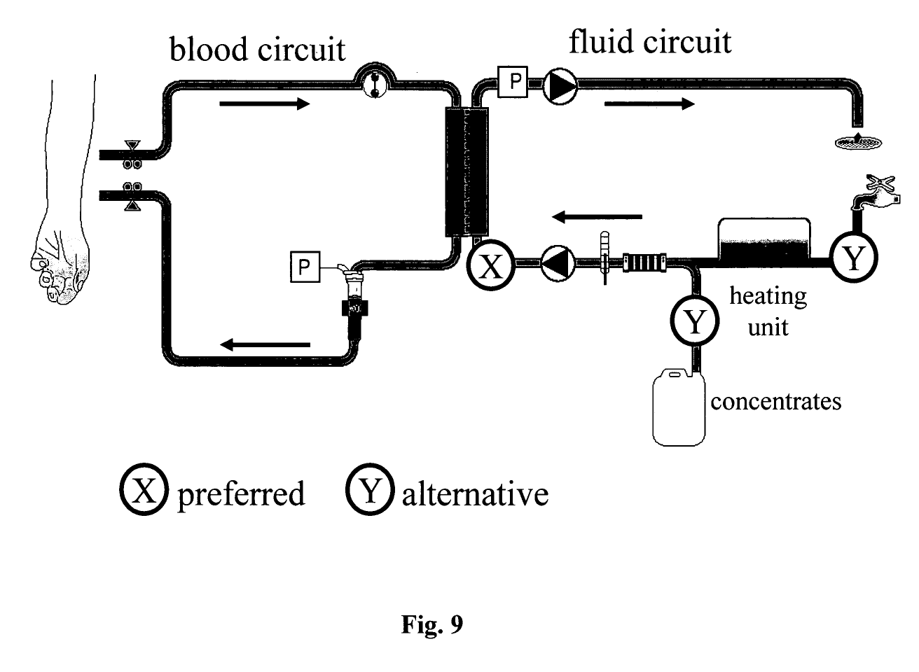

FIG. 9 shows schematically how the invention can be applied in a typical dialysis process. The scheme shows the principle of the hemodialysi process, i.e. a fluid circuit contained in a dialysis machine and the blood circuit with a dialyzer connected to a patient. RO water coming from a distribution pipe enters the dialysis machine, where electrolytes are admixed (either from liquid concetrates or solid powders) to prepare the dialysate. Dialysate is then pumped through the dialysate compartment of the dialyzer. On the blood circuit side patient's blood is pumped through the blood compartment of the dialyzer and returned to the patient. The circles indicate the possible locations of means of the invention to reduce the DNA content of water or dialysate.

FIGS. 10-13 show examples of how the devices of the invention can be used to remove the contaminating oligonucleotides in the therapeutic fluids. The exact location of the device(s) can be adjusted depending on a given application. The device(s) can be used singly or multiply.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### EXAMPLES

### Example 1

Bacterial Culture

Clinical isolates of Pseudomonas aeruginosa were grown in standard dialysate, e.g. bicarbonate hemofiltration fluid such as Schiwa HF Bic 35 410. The bacteria were pelleted by centrifugation (2000 g for 30 min) and the culture supernatant was filtered through 0.45 μm cellulose acetate filters (Nalgene, United States Palstics Corp. Lima, Ohio) to remove any residual bacteria.

### Example 2

Peripheral Blood Mononuclear Cell (PBMC) Preparation

PBMCs were separated from whole blood by centrifugation through Ficoll and Hypaque made from powder (Ficoll Type 400; sodium-diatrizoate, Sigma). The water for preparation of Ficoll and Hypaque and all other fluids used were subjected to ultrafiltration using polyamide filters (PF14S, Gambro, Hechingen, FRG) to remove cytokine-inducing substances. For incubation, PBMCs were washed twice with normal saline, resuspended at 5×10⁶/ml in serum-free RPMI 1640 culture medium (Gibco, Paisley, UK), supplemented with 2 mM L-glutamine, 100 U/ml penicillin and 100 mg/ml Streptomycin.

### Example 3

Incubation Of Bacterial Filtrates With Beads

50 ml of bacterial filtrate was incubated with 1 g of sterilized beads (autoclaved at 121° C., 20 min, these beads were those described in U.S. Pat. No. 6,255,477) for 2 h at room temperature on a rocking platform. The supernatant was obtained after centrifugation (1000 g, for 30 min).

### Example 4

Incubation Of Isolated PBMC With Bacterial Filtrates

250 μl of cell suspension were incubated in 24-well plates (Nunc, Denmark) for 18 hours with 250 μl of bacterial filtrate (diluted 1:10) at 37° C. in a humidified atmosphere containing 5% CO₂. After incubation, PBMCs were subjected to three freeze/thaw cycles to lyse the cells.

### Example 5

Cytokine Assays

IL-1β and IL-6 were measured in the lysed cells after two freeze/thaw cycles by ELISA. Primary and biotinylated antibodies against IL-1β and IL-6 were purchased from R&D Systems (R&D, Wiesbaden, Germany). 96-well plates (Maxisorp, Nunc, Denmark) were coated overnight with 50 μl/well of the primary antibody in coating buffer (0.2 M NaHCO₃/Na₂CO₃, pH 10.5). Wells were blocked with 0.2% casein (Sigma) in PBS for one hour, 50 μl of cytokine standards or samples were added to the wells and incubated overnight. All dilutions were made in PBS containing 0.05% Tween (Sigma); after each incubation step; wells were washed with PBS containing 0.05% Tween. 50 μl/well of the appropriately diluted biotinylated secondary antibody was added and incubated for 1 hour. After incubation with peroxidase-Streptavidin-biotin complexes (Amersham, Braunschweig, Germany) for one hour, plates were developed with TMB (240 μg/ml 3,3′,5,5′ tetramethylbenzidine, Fluka Chemicals, Buchs, Switzerland) in Gallati buffer (42 μg/ml citric acid, pH 3.95/0.01% H₂O₂). Optical density was determined at 450 and 630 nm on an ELISA plate reader (Dynastar MR5000). The sensitivity of the assays varied between 10 and 30 μg/ml for IL-1β and 5 to 10 μg/ml for IL-6. Samples were measured in at least two dilutions until their concentrations were in the linear part of the standard curve.

When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomers and enantiomer of the compound described individual or in any combination. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application.

Whenever a range is given in the specification, for example, a temperature range, a time range, or a composition or concentration range, all intermediate ranges and subranges, as well as all individual values included in the ranges given are intended to be included in the disclosure.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, reagents, solid substrates, synthetic methods, purification methods, and analytical methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference to the extent that there is no inconsistency with the disclosure of this specification. Some references provided herein are incorporated by reference to provide details concerning sources of starting materials, additional starting materials, additional reagents, additional methods of synthesis, additional methods of analysis and additional uses of the invention.

1. A device for purifying a therapeutic fluid wherein the device comprises a means for eliminating or reducing oligonucleotides from the fluid.

2. The device of claim 1 wherein the means is an ultrafiltration means.

3. The device of claim 1 wherein the means is an adsorption means.

4. The device of claim 1 wherein the means is a combination of ultrafiltration means and absorption means.

5. The device of claim 2, wherein said ultrafiltration is carried out by a membrane comprising positively charged material.

6. The device of claim 5, wherein the positively charged material is a polyarylethersulfone and polyethyleneimine (PAES/PEI) blend or a polyarylethersulfone and polyphenyleneoxide (PAES/PPO) blend.

7. The device of claim 2 wherein said ultrafiltration is carried out by a membrane comprising nanoparticles of glass composites.

8. The device of claim 3 wherein the oligonucleotides are removed by adsorbing onto beads comprising a composition selected from the group consisting of polystyrene grafted with polyethyleneglycol (PEG), polyarginine, ARG-8, glass composite particles and collagen coated matrices.

9. The device of claim 1 wherein the oligonucleotide is of microbial origin.

10. The device of claim 9 wherein the means is capable of removing said oligonucleotides ranging in size from as about 5 nucleotides up to about 500 nucleotides.

11. The device of claim 10 wherein the means is capable of removing said oligonucleotides ranging in size from as about 5 nucleotides up to about 200 nucleotides.

12. The device of claim 10 wherein the fluid is peritoneal dialysis fluid.

13. A process for purifying a therapeutic fluid wherein the process comprises a means of eliminating or reducing oligonucleotides from the fluid.

14. The process of claim 13 wherein the oligonucleotides are removed by ultrafiltration.

15. The process of claim 13 wherein the oligonucleotides are removed by adsorption.

16. The process of claim 13 wherein the oligonucleotides are removed by a combination of ultrafiltration and adsorption.

17. The process of claim 13 wherein the ultrafiltration is carried out by a membrane comprising positively charged material.

18. The process of claim 17 wherein the positively charged material is a polyarylethersulfone and polyethyleneimine (PAES/PEI) blend or a polyarylethersulfone and polyphenyleneoxide (PAES/PPO) blend.

19. The process of claim 13 wherein said ultrafiltration is carried out by a membrane comprising nanoparticles of glass composites.

20. The process of claim 15 wherein the oligonucleotides are removed by adsorbing onto beads comprising a composition selected from the group consisting of polystyrene grafted with polyethyleneglycol (PEG), polyarginine, ARG-8, glass composite particles and collagen coated matrices.

21. The process of claim 20 wherein the oligonucleotide is of microbial origin.

22. The process of claim 21 wherein the means is capable of removing said oligonucleotides ranging in size from about 5 nucleotides up to about 500 nucleotides.

23. The process of claim 22 wherein the means is capable of removing said oligonucleotides ranging in size from about 5 nucleotides up to about 200 nucleotides.

24. The process of claim 22 wherein the fluid is peritoneal dialysis fluid.
